(19) United States
(12) Patent Application Publication
(10) Pub. No.: US 2008/0228286 A1
(43) Pub. Date: Sep. 18, 2008

(54) Liner

(75) Inventor: Cornelis Dirk Jongenengel, Poortugaal (NL)

Correspondence Address: SWANSON & BRATSCHUN, L.L.C., 8210 SOUTHPARK TERRACE, LITTLETON, CO, 80120, US

(73) Assignee: Jongenengel Research & Development B.V., Rotterdam (NL)

(21) Appl. No.: 10/598,254

(22) PCT Filed: Feb. 23, 2005
(86) PCT No.: PCT/NL2005/000133

§ 371 Date: Sep. 12, 2007

(30) Foreign Application Priority Data
Feb. 23, 2004 (NL) 1025552

(51) Int. Cl. A61F 2/78

(52) U.S. Cl. 623/36

(57) ABSTRACT

The invention relates to a liner for connecting a prosthesis with the stump of an amputated lower leg. The liner comprises a stocking from a substantially flexible material for surrounding the stump with a first, open end for receiving the stump there through and a second, closed end which is, on the outside, provided with coupling means for coupling to the prosthesis. The liner is, on the inside, provided with a substantially form-retaining disc for supporting the end of the stump. Further, the substantially form-retaining disc is substantially non-round in a view from the open end of the stocking.

The invention relates to a liner for connecting a prosthesis with the stump of an amputated lower leg, comprising a stocking from a substantially flexible material for surrounding the stump with a first, open end for receiving the stump therethrough and a second, closed end which is, on the outside, provided with coupling means for coupling to the prosthesis, where the stocking is, on the inside, provided with a substantially form-retaining disc for supporting the end of the stump.

Such liners are known per se and offer persons with an amputated lower leg the possibility to firmly attach a prosthesis, such as an artificial limb, to the stump of the lower leg for supporting the stump.

When applying the liner, the user slides the stump through a first, open end of the stocking, until the end of the stump abuts the form-retaining disc having a circular circumferential edge. Then, the stump with the stocking can be received in a sleeve-shaped, stiff part of the prosthesis and be coupled to the prosthesis via the coupling means.

The stocking is applied so as to be rough and substantially airtight with respect to the skin of the stump, so that sliding and/or coming loose of the stocking and the prosthesis coupled thereto is prevented. Because the stocking has been applied firmly, the disc also remains in predetermined position relative to the end of the stump. Since the disc is substantially form-retaining and abuts the end of the stump, it forms a support of the lower part of the stump. The prosthesis can thus be attached to the lower leg with relatively little play, which is favorable to the ease of use. Due to the stiffness of the material at the end of the stump, the substantially form-retaining disc can cause irritations and/or pain symptoms. By contrast, use of materials with a greater flexibility can result in a less firm attachment of the prosthesis to the lower leg, which results in a less adequate support of the stump and is therefore undesired.

The invention relates to a liner according to the introduction, where, while retaining the advantages, the disadvantages mentioned are avoided. In particular, the invention contemplates obtaining a liner, where the creation of irritations and/or pain symptoms at the end of the stump is prevented, while no concessions are made to the supporting function of the disc and the firmness of the connection between the prosthesis and the stump. For this purpose, the substantially form-retaining disc is substantially non-round in a view from the open end of the stocking.

By designing the substantially form-retaining disc so as to be substantially non-round in the view from the open end, it is achieved that the disc can more suitably follow the anatomical shape of a cross section of the lower leg near the end of the stump, while still a good connection of the disc with the lower surface of the stump can be realized. This increases the wearing comfort of the prosthesis with a similar or even improved supporting function of the disc, so that pain symptoms and irritations at the end of the stump do not occur easily. A substantially non-round shape is understood to mean that, in the view from the open end of the stocking, the disc has a substantially smoothly flowing circumferential edge whose radius of curvature varies.

Preferably, the substantially form-retaining disc is substantially oval in the view from the open end of the stocking, so that, with an oval or egg-shaped profile, the disc can more suitably follow the anatomical shape of the cross section of the part of the lower leg to be supported, so that the wearing comfort improves.

By designing the form-retaining disc so as to be substantially triangular in the view from the open end of the stocking, symptoms of the stump of the lower leg are reduced even further. This is based on the insight that the outer profile of the lower leg is substantially more triangular than round due to the interior structure. By designing the disc to be substantially triangular as well, the anatomical shape of the cross section of the part of the lower leg to be supported can therefore be followed even more suitably. In addition, due the substantially triangular shape of the container, the supporting surface under the stump is reduced, which is further favorable to the comfort of the user. Preferably, the corners of the substantially triangular shape are rounded, so that a better connection to the end of the stump is realized, which is favorable to the wearing comfort.

Preferably, the form-retaining disc is attached to the coupling means via a force-transmitting element, so that, during use of the liner, forces are transmitted from the prosthesis via the coupling means and the force-transmitting element to the form-retaining disc for supporting the stump.

Further advantageous embodiments of the invention are described in the subclaims.

The invention will be explained in more detail with reference to an exemplary embodiment shown in the drawing, in which:

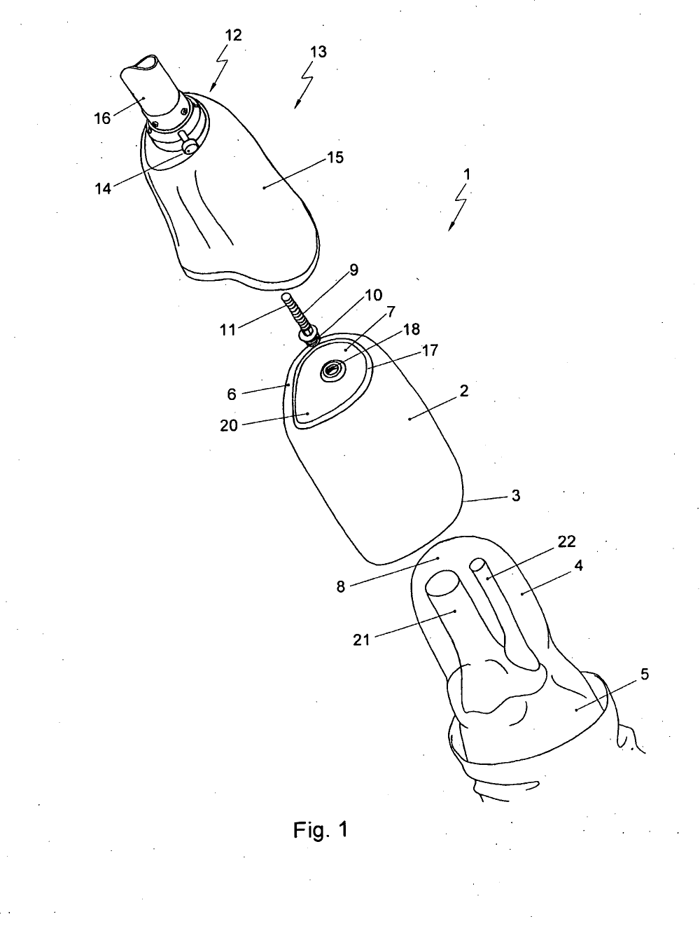

FIG. 1 shows a diagrammatic perspective view of a liner in exploded condition according to the invention;

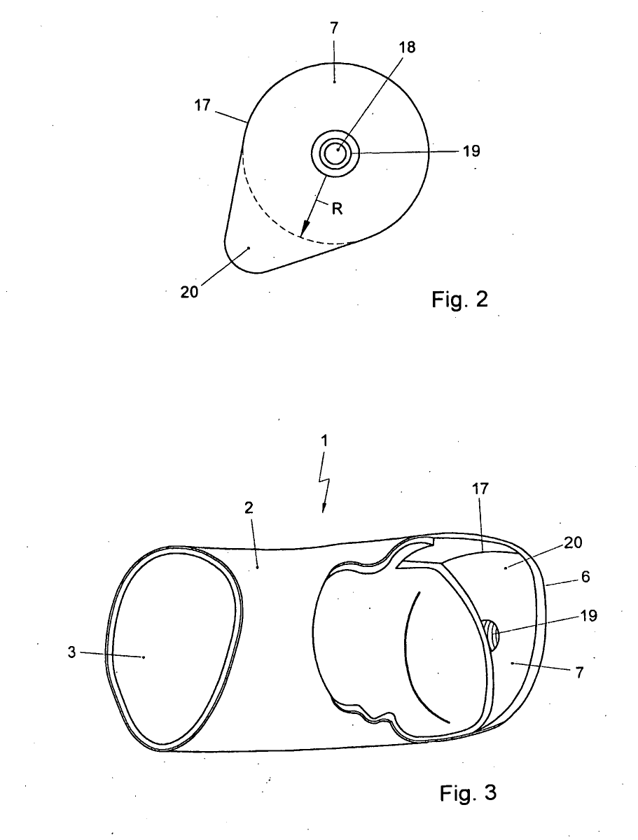

FIG. 2 shows a diagrammatic top plan view of a disc of the liner of FIG. 1; and

FIG. 3 shows a diagrammatic perspective view of a partly cutaway liner of FIG. 1.

The Figures are only diagrammatic representations of a preferred embodiment of the invention. In the Figures, same or corresponding parts are designated by the same reference numerals.

FIG. 1 shows a preferred embodiment of a liner 1 according to the invention. The liner 1 comprises a stocking 2 which is built up from a flexible and skin-friendly material, for instance a silicone material. The stocking 2 has a first, open end 3, through which the stump 4 of an amputated lower leg 5 can be received. As is shown in more detail in the partly cutaway liner 1 of FIG. 3, the second end 6 of the stocking 2 is closed. On the inside of the stocking 2, at the end, a cup-shaped disc 7 has been provided, on which the end or lower surface 8 of the stump 4 operatively rests. Further, the second end 6 is, on the outside, provided with coupling means, designed as coupling pin 9, which extends, by a first end 10, into an opening 18 in the disc 7 and is fixed with screw thread 19, and is, by a second end 11, operatively coupled with a blocking system 12 of a prosthesis 13, for instance with the aid of a ratchet mechanism. The coupling with the blocking system 12 can be released by pressing a decoupling pin 14, which has been provided to the prosthesis 13. With the aid of the coupling means, the forces are transmitted from the prosthesis 13 to the disc 7. Then, the disc 7 transmits the forces to the stump, as will be set forth in the following.

When applying the prosthesis 13, first of all, the liner 1 is slid over the stump 4 so far that the disc 7 abuts the lower surface 8 of the stump 4. Here, the stocking 2 airtightly surrounds the stump 4 with the flexible material, so that the liner 1 firmly surrounds the lower leg 5 and more or less fixes the position of the disc 7 relative to the stump 4. Then, the prosthesis 13 can be coupled to the disc 7 with the coupling means.

The prosthesis 13 is an artificial limb or lower leg and has a sleeve-shaped, stiff part 15, also referred to as container, which has been pre-formed as a cover part in order to connect properly to the stump 4 surrounded by the stocking 2. The container 15 is attached to the prosthesis 13 near the blocking system 12 and extends in a first direction relative to the blocking system 12. The prosthesis 13 further comprises a construction 16 which extends in a different direction relative to the blocking means 12 and to which other parts of the artificial limb are attached, such as an ankle joint and an artificial foot. During use, the container 15 has been slid over the stocking 2 around the stump 4 so far that the coupling means can engage the blocking system. The container 15 is built up from a material composition which is deformable a single time, for instance by heating or by contact with a substance comprising specific chemical components. During the deformation, the container 15 is applied around the stump 4 surrounded by the stocking 2, so that a desired anatomical shape is obtained. After the deformation, the container 15 assumes a final shape.

The disc 7 is designed as a cup, slightly hollow, so that the surface comfortably connects to the curvature of the lower surface 8 of the stump 4. As shown in FIG. 3, the disc 7 is non-round in a view from the open end of the stocking, i.e. the circumferential edge flows smoothly and has a varying radius of curvature R. In one segment of the circumferential edge 17, the radius of curvature is relatively small, so that a slightly triangular profile with rounded corners is obtained. In the segment with the relatively small radius of curvature, a relatively acute angle 20 of the triangular profile is created. In an advantageous manner, with a part located near the relatively acute angle 20, the disc 7 supports the tibia 21, so that the supporting forces are transmitted via a relatively large surface to the bone structure of the amputated lower leg 5 in an anatomically comfortable manner. As can be seen in FIG. 2, the fibula 22 is also supported by the substantially cup-shaped disc 7.

The invention is not limited to the above-described exemplary embodiments. Many variants are possible.

For instance, the coupling means may also be designed in a different manner, for instance with the aid of a snap system.

Further, the final shape of the container may be realized in a different manner, for instance via a series of mechanical operations.

Such variants will be clear to a skilled person and are understood to be within the scope of the invention as set forth in the following claims.

1. A liner for connecting a prosthesis with the stump of an amputated lower leg, comprising a stocking from a substantially flexible material for surrounding the stump with a first, open end for receiving the stump there through, and a second, closed end which is, on the outside, provided s with coupling means for coupling to the prosthesis, wherein the stocking is, on the inside, provided with a substantially form-retaining disc for supporting the end of the stump, and wherein the substantially form-retaining disc is substantially non-round in a view from the open end of the stocking.

2. The liner according to claim 1, wherein the substantially form-retaining disc is substantially oval in the view from the open end of the stocking.

3. The liner according to claim 1, wherein the substantially form-retaining disc is substantially triangular in the view from the open end of the stocking.

4. The liner according to claim 3, wherein the corners of the substantially triangular shape are rounded.

5. The liner according to claim 3, wherein the substantially triangular disc is arranged for supporting the tibia with a part which is located near a corner of the triangle.

6. A liner according to claim 3, wherein the substantially triangular disc has a substantially cup-shaped design.
